(19) United States
(12) Patent Application Publication
(10) Pub. No.: US 2023/0149450 A1
(43) Pub. Date: May 18, 2023

(54) PREPARATION FOR MAGNETIZING KIDNEY STONES AND KIDNEY STONE FRAGMENTS AND KIT FOR REMOVING KIDNEY STONES AND KIDNEY STONE FRAGMENTS

(71) Applicant: Technische Universität München, München (DE)

(72) Inventors: Sebastian Patrick SCHWAMINGER, Cambridge, MA (US); Shyam SRINIVASAN, München (DE); Laura Annabelle SOLVIE, München (DE); Camilo TOLOZA, Jena (DE); Michael WENGLER, Garching (DE)

(21) Appl. No.: 17/802,655

(22) PCT Filed: Feb. 23, 2021
(86) PCT No.: PCT/EP2021/054434

§ 371 Date: Aug. 26, 2022

(30) Foreign Application Priority Data
Mar. 2, 2020 (WO) 20160490.7

(51) Int. Cl. A61K 33/26; A61K 33/42; A61K 9/10; A61K 9/14; A61K 47/54; A61K 47/62

(52) U.S. Cl. CPC A61K 33/26; A61K 33/42; A61K 9/10; A61K 9/14; A61K 47/549; A61K 47/62

(57) ABSTRACT

A preparation for magnetizing kidney stones or kidney stone fragments contains magnetic particles, at least one salt selected from the group consisting of: chlorides, perchlorates and phosphates of Li, Na, K, ammonium, Mg and Ca, and at least one solvent. The preparation is in the form of a solution, a suspension, or a dispersion.

The present invention relates to a preparation for magnetizing kidney stones or kidney stone fragments, which enables kidney stones or kidney stone fragments to be easily removed from the kidney of a human body. Furthermore, the invention also relates to a kit for removing kidney stones or kidney stone fragments.

Kidney stones are crystalline deposits that can form in the kidney and also in the urinary tract of a mammal. Current studies show that around 12% of the world's population suffers from kidney stones, which are very painful and can lead to secondary damage. Often there is no other way than to surgically remove the kidney stones. The kidney stones, depending on their size, are first broken up, for example using laser pulses, before removal. The disintegrated kidney stones are then extracted through the ureter using a snare. The disadvantage of this procedure is that smaller kidney stone fragments can easily be overlooked, which leads to renewed crystal growth and the formation of further kidney stones. A kit for removing kidney stones is known from U.S. Pat. No. 9,925,311 B2, which comprises magnetic or magnetizable particles, a crosslinkable polymer and a crosslinking agent. In this case, the magnetic or magnetizable particles bind to the kidney stones, and the crosslinkable polymer forms a kind of gel through crosslinking with the crosslinking agent, which binds the kidney stones magnetized by the magnetic or magnetizable particles so that they can be discharged from the body. The disadvantage here is that the provision of this kit is very expensive. In addition, the preparation for removing kidney stones contains fewer physiological components such as the crosslinking polymer and the crosslinking agent, which can lead to intolerance and damage to the kidney. Also, the use of the crosslinking polymer requires a very low pH of around 3.5, which makes it difficult for the user to handle the preparation.

Based on this prior art, it is an object of the present invention to provide a preparation for magnetizing kidney stones or kidney stone fragments which allows kidney stones or kidney stone fragments to be removed safely and easily from the kidney of a human body, avoids consequential damage and diseases, and is easily handled by the user.

A further object of the invention is to provide a kit which is easy and safe to use and with which both kidney stones and kidney stone fragments can be removed easily and completely.

The object is achieved by a preparation for magnetizing kidney stones or kidney stone fragments, containing magnetic particles, at least one salt selected from the group consisting of: chlorides, perchlorates and phosphates of lithium (Li), sodium (Na), potassium (K), ammonium (NH₄⁺), magnesium (Mg) and calcium (Ca), such as in particular LiCl, NaCl, KCl, NH₄Cl, MgCl₂ and CaCl₂, and at least one solvent. The preparation is in the form of a solution, a suspension or a dispersion. When using the preparation according to the invention, the use of magnetic particles makes it possible to magnetize the kidney stones or kidney stone fragments (in the following, kidney stones and kidney stone fragments are summarized under the term “kidney stones”), so that they can be removed from a patient's kidneys using a magnetic needle, for example. The kidney stones to be magnetized using the preparation according to the invention are not restricted in detail and they can be, for example, calcium oxalates, calcium phosphates, struvite stones (ammonium magnesium phosphate), urine stones or cystine stones.

In the context of the present invention, magnetic particles are understood to mean particles which can be attracted by a magnet due to magnetic interaction. Here, the particles used according to the invention are not limited in detail and can have different chemical compositions. Preferred magnetic particles here are magnetites, maghemites and ferrites, ferrites being understood to mean in particular compounds of the XFe₂O₃ type, where X═Co, Cu, Ni, Zn, Mn, Ba, Sr and Mg.

Surprisingly, it was found that the kidney stones and, in particular, also very small fragments can be completely removed by using at least one of the salts mentioned above, or else mixtures of two or more of the salts. The salt prevents or suppresses the electrostatic repulsion of the magnetic particles, so that there is improved particle aggregation of the kidney stones magnetized by the magnetic particles. The kidney stones can thus be completely removed from a kidney very safely and easily. The magnetized kidney stones thus do not tend to disperse in the kidney, but completely aggregate by reducing the repulsive interactions between the magnetic particles, without the need for chemical aggregating agents such as cross-linking polymers.

The preparation according to the invention is thus distinguished by very good tolerability, simple and safe handling, and an inexpensive composition. Due to the salt used, it can be provided in the form of a solution, suspension or dispersion, even without a preservative, and this makes it easier to use since the preparation does not have to be prepared in a complicated manner. The preparation can thus be provided as required.

The dependent claims relate to preferred embodiments and refinements of the invention.

Due to their very good availability, the magnetic particles are preferably ferromagnetic particles, and in particular are selected from iron, nickel, cobalt, AlNiCo, SmCo, Nd₂Fe₁₄B, Ni₈₀Fe₂₀, NiFeCo, ferrites, Fe₃O₄, gamma-Fe₂O₃, mixed phases of Fe₃O₄ and gamma-Fe₂O₃, and mixtures thereof. Ferrites, Fe₃O₄, gamma-Fe₂O₃, mixed phases of Fe₃O₄ and gamma-Fe₂O₃ and mixtures of these iron oxides are particularly preferred because they have very high magnetizations in magnetic fields, and thus can be very easily attracted magnetically. In addition, these magnetic particles are inexpensive particles that can be obtained in any particle size, and are characterized by good compatibility, i.e., no appreciably toxic character. In particular, mixtures of metallic particles such as iron, nickel, cobalt, AlNiCo, SmCo, Nd₂Fe₁₄B, Ni₈₀Fe₂₀ and NiFeCo with one or more of the above metal oxides can also be used.

Due to the very good dispersibility in the preparation according to the invention, the primary particle size of the magnetic particles is 2 to 100 nm, in particular 4 to 20 nm, and in particular 5 to 15 nm. The smaller the particle size, the better the distribution of the magnetic particles in the preparation, and thus also in the kidneys of a patient. However, this also has the disadvantage that aggregation can be made more difficult. In light of the above viewpoints, the primary particle size of the magnetic particles is preferably 5 to 15 nm. The primary particle size is determined here by means of transmission electron microscopy.

According to a further preferred development, the magnetic particles have a specific surface area of from 80 to 150 m²/g, and in particular from 90 to 120 m²/g. This improves binding to the kidney stones. The magnetic particles surround the kidney stones and thus facilitate their aggregation. The specific surface area is determined here by means of gas adsorption isotherms according to the BET method using nitrogen at 77 K.

In order to improve the long-term stability of the preparation and to facilitate the distribution of the preparation in a patient's kidneys using conventional injection devices, the concentration of the magnetic particles, based on the preparation, is 2 to 40 g/L, and in particular 4 to 20 g/L.

The repulsive interaction between the magnetic particles can be reduced particularly well and even almost completely suppressed if the concentration of the salt in the preparation is preferably at least 100 mmol, in particular 100 to 2000 mmol, and in particular 200 to 1000 mmol. A concentration in the range from 200 to 1000 millimolar is particularly preferred for reasons of physiological compatibility, and also for reasons of cost.

Also for reasons of cost and for reasons of very good compatibility, the solvent is preferably water. The use of alcohol or mixtures of water and alcohol can be advantageous in terms of improved aggregation, since the OH— groups of the alcohol or alcohols make it easier for the kidney stones to bind to the magnetic particles. The alcohol is not restricted in any way; but ethanol and glycerin are particularly preferred.

A further advantageous development provides that the preparation further contains at least one sugar and/or at least one protein and/or at least one physiological buffer. An addition of sugar and/or protein has the advantageous effect that the viscosity of the preparation is increased, so that the aggregation of magnetized kidney stones and their removal from the kidney are facilitated.

For reasons of physiological compatibility and for reasons of user safety, the pH of the preparation is preferably in a range from 5 to 8, and in particular from 6 to 7. This is possible in particular because the preparation according to the invention, due to its simple chemical composition, can dispense with other auxiliaries that require an acidic or basic pH, such as, for example, surfactants, aggregating agents, and the like.

In particular, the preparation is substantially free of film-forming and crosslinking polymers for reasons of cost savings and physiological compatibility. This means that no crosslinking polymers or crosslinking agents for these polymers are added to the preparation.

Likewise according to the invention, a kit for removing kidney stones or kidney stone fragments is also described. The kit comprises the preparation disclosed above and a magnetic or magnetizable device by means of which the kidney stones or kidney stone fragments magnetized by the preparation according to the invention can be attracted and removed from a patients kidney. Particularly suitable devices are magnetic needles which comprise a permanent magnetic material or an electromagnetic material with which the magnetized and aggregated kidney stones can interact and be reversibly bound. A complete removal of kidney stones from a patients kidney is very safe and easy.

Further details, features and advantages of the invention result from the following description and the figures, wherein:

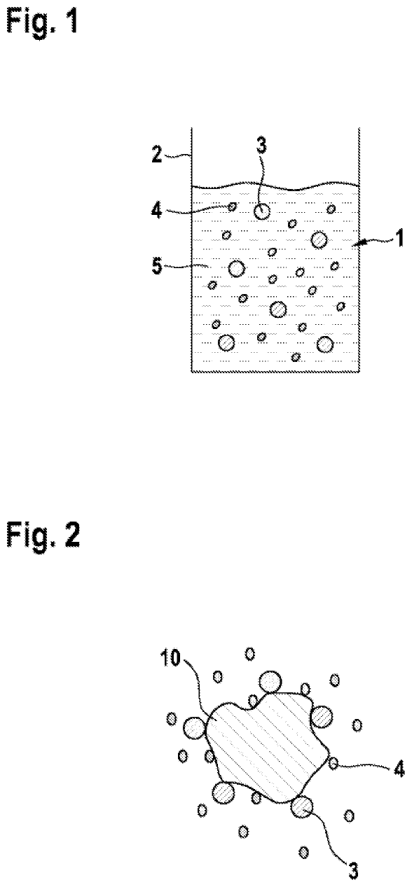

FIG. 1 is a schematic view of a preparation according to an embodiment of the invention,

FIG. 2 is a schematic sectional view of a kidney stone treated with the preparation from FIG. 1, and

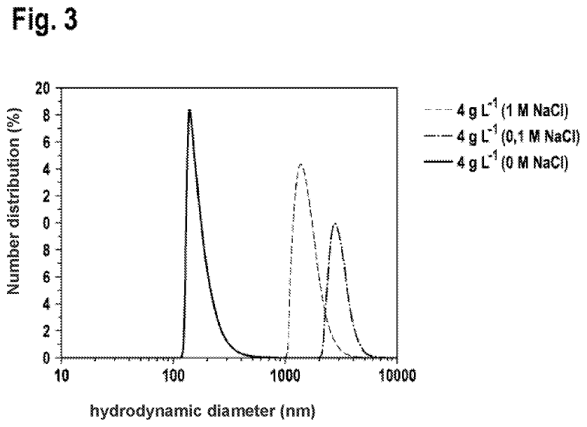

FIG. 3 is a diagram illustrating the hydrodynamic diameter of magnetic particles as a function of the concentration of NaCl.

Only the essential features of the invention are shown in the figures. All other features have been omitted for the sake of clarity. In addition, the same reference symbols designate the same components.

In detail, FIG. 1 shows a preparation 1 contained in a vessel 2 for magnetizing kidney stones or kidney stone fragments. The preparation 1 can be used, for example, in a surgical procedure in which the preparation 1 is introduced into a patient's kidney.

The preparation 1 is, for example, in the form of a suspension, and thus contains a solvent 5 and magnetic particles 3 contained therein, and a salt 4 which is selected from the group consisting of: chlorides, perchlorates and phosphates of Li, Na, K, ammonium, Mg and Ca, such as in particular LiCl, NaCl, KCl, NH₄C1, MgCl₂ and CaCl₂.

The salt 4 used is preferably NaCl, which is usually present in dissolved form in the solvent 5, which is preferably water. However, since FIG. 1 is an illustrative representation, the salt 4 is shown in particulate form.

As FIG. 1 shows, the magnetic particles 3 are distributed homogeneously in the preparation 1. There are only slight repulsive interactions between the magnetic particles 3. The repulsive interactions are suppressed by the salt 4. More importantly, the magnetic particles 3 are held together spatially by the electrostatic interaction with the salt 4, but without clumping.

The preparation 1 is characterized by high long-term stability, is safe to use and, as shown schematically in FIG. 2, enables kidney stones and kidney stone fragments to be magnetized. In addition, no further aggregation means are required to enable kidney stones and even very small kidney stone fragments to be completely removed from a kidney.

FIG. 2 shows how the magnetic particles 3 bind to the kidney stone 10. This bond is in particular covalent, but can also be of a physical nature. As soon as the magnetic particles 3 get close to a kidney stone 10 by means of the preparation 1, the magnetic particles 3 bind to the surface of the kidney stone 10, so that the kidney stone 10 is “magnetized.” An aggregation of several kidney stones 10 magnetized in this way is achieved by the presence of the salt 4, which reduces or even suppresses the repulsive interactions acting between the magnetic particles 3 and thus enables the magnetized kidney stones 10 to be attracted electrostatically. Thus, the magnetized kidney stones 10 can be very easily attracted and removed from the kidney by a magnetic device that can also be inserted into the kidney.

FIG. 3 is a graph illustrating the hydrodynamic diameter of magnetic particles as a function of the concentration of NaCl. The hydrodynamic diameter is a measure of the ability of the magnetic particles to aggregate. The larger the hydrodynamic diameter, the better the ability to aggregate and the easier it is for kidney stones to be magnetized and extracted from a kidney after aggregation.

Fe₃O₄ particles were used as the magnetic particles. The nanoparticles used here were produced in a co-precipitation of iron(II) chloride and iron(III) chloride with caustic soda according to the Massart process (see, for example: Roth et al. https://doi.org/10.1016/j.jmmm.2014.10.074). The particles were washed with deionized water and then freeze-dried for X-ray diffraction, nitrogen adsorption isotherms according to the BET method, and magnetometry. The TEM measurements revealed an average particle diameter of 10 nm, and the X-ray diffraction pattern verified magnetite as the main structure of the particles. For the transmission electron microscopy, the samples were dripped in a concentration of approx. 0.01 g/L onto Quantifoil TEM grids (10 μL), which had previously been hydrophilized using air plasma for 30 s. The samples were dried with a cold air dryer and measured, after a further day, in a JEOL JEM 1400Plus transmission electron microscope. The TEM was calibrated using the diffraction pattern of a catalase crystal. At least 5 images were taken of the iron oxide samples, and at least 30 particles per image were measured using the image processing program ImageJ for statistical analysis of the particle size. A primary particle diameter of 9 nm could be determined with X-ray diffraction using the Scherrer method. The magnetometric measurements revealed a saturation magnetization of 75 Am²/kg in a field of 50000 Oe. In addition, no remanence and therefore superparamagnetic behavior could be detected in the particles. The specific surface area of the particles according to the BET method with nitrogen at 77 K was 100 m²/g. A Gemini VII from Micromeritics was used for this, and 9 points between 0.05 and 0.25 p/P₀ were measured. The dead volume of the sample was determined beforehand with helium at 77 K.

The particles had a hydrodynamic diameter of 180 nm and an isoelectric point at pH 6. These magnetic particles were dispersed in water as a solvent at a concentration of 4 g/L.

In this way, three suspensions were prepared, each of which was filled into a vessel. NaCl was not added as a salt to vessel 1 (concentration of NaCl: 0 M). NaCl was added to vessel 2 so that the concentration of NaCl in the preparation was 0.1M. NaCl was added to vessel 2 so that the concentration of NaCl in the preparation was 1M.

The diagram shows that the hydrodynamic diameter increases with increasing NaCl concentration. Thus, the ability to aggregate kidney stones magnetized by means of the preparation also increases, since the repulsive interaction between the magnetic particles is reduced.

### LIST OF REFERENCE SIGNS

1 preparation

2 vessel

3 magnetic particles

4 salt

5 solvent

10 kidney stone

1. A preparation for magnetizing kidney stones or kidney stone fragments, containing:
magnetic particles,
at least one salt selected from the group consisting of: chlorides, perchlorates and phosphates of Li, Na, K, ammonium, Mg and Ca, and
at least one solvent,
wherein the preparation is in the form of a solution, a suspension or a dispersion.

2. The preparation according to claim 1, wherein the magnetic particles are ferromagnetic particles, and in particular are selected from iron, nickel, cobalt, AlNiCo, SmCo, Nd₂Fe₁₄B, Ni₈₀Fe₂₀, NiFeCo, ferrites, Fe₃O₄, gamma-Fe₂O₃, mixed phases of Fe₃O₄ and gamma-Fe₂O₃, and mixtures thereof.

3. The preparation according to claim 1, wherein the magnetic particles have a primary particle size of 2 to 100 nm.

4. The preparation according to claim 1, wherein the magnetic particles have a specific surface area of from 80 to 150 m²/g.

5. The preparation according to claim 1, wherein the concentration of the magnetic particles, based on the preparation, is 2 to 40 g/l.

6. The preparation according to claim 1, wherein the concentration of the salt in the preparation is at least 100 mmol.

7. The preparation according to claim 1, wherein the solvent is selected from water, alcohol, and mixtures thereof.

8. The preparation according to claim 1, further containing at least one sugar and/or at least one protein and/or at least one physiological buffer.

9. The preparation according to claim 1, wherein the pH of the preparation is in a range from 5 to 8.

10. The preparation according to claim 1, wherein the preparation is substantially free of film-forming and crosslinking polymers.

11. A kit for removing kidney stones or kidney stone fragments, comprising the preparation according to claim 1, and a magnetic or magnetizable device, in particular a magnetic needle, for removing kidney stones or kidney stone fragments magnetized by the preparation.

12. The preparation according to claim 1, wherein the magnetic particles have a primary particle size of 4 to 20 nm.

13. The preparation according to claim 1, wherein the magnetic particles have a primary particle size of 5 to 15 nm.

14. The preparation according to claim 1, wherein the magnetic particles have a specific surface area of from 90 to 120 m²/g.

15. The preparation according to claim 1, wherein the concentration of the magnetic particles, based on the preparation, is 4 to 20 g/l.

16. The preparation according to claim 1, wherein the concentration of the salt in the preparation is 100 to 2000 mmol.

17. The preparation according to claim 1, wherein the concentration of the salt in the preparation is 200 to 1000 mmol.

18. The preparation according to claim 1, wherein the pH of the preparation is in a range from 6 to 7.
